(19) United States
(12) Patent Application Publication
(10) Pub. No.: US 2012/0059387 A1
(43) Pub. Date: Mar. 8, 2012

(54) Delivery system and urethra stent for enlarged prostates and method

(75) Inventors: Richard W. Schanz, St. George, UT (US); J. Bryan Ellsworth, St. George, UT (US)

(73) Assignee: Schanz II, LLC

(21) Appl. No.: 12/807,386

(22) Filed: Sep. 3, 2010

(51) Int. Cl. A61B 17/00

(52) U.S. Cl. 606/108

(57) ABSTRACT

A device for placing, positioning and/or removing a stent within the urethra of a patient obstructed by an enlarged prostate positioned by use of ultrasound comprising a flexible stent inserter having a shaft of a length and size to be inserted within the urethra and extend to the bladder, with a balloon positioning tip distally mounted proximate an expandable securing segment onto which a stent is mounted; the balloon selectively inflated with air or liquid if a contrasting media is required for ultrasound location or with a liquid to unblock obstructed urethra segments, and the securing segment selectively inflated with a liquid to secure during positioning and deflated to release the stent in position to inflate the expandable securing segment to hold the stent during positioning and insertion, and in a second mode deflates the securing segment and positioning tip to release the stent within the urethra to allow the flexible stent inserter to be withdrawn and allow urine to pass through the stent.

### BACKGROUND OF THE INVENTION

1. Field of Invention

This invention pertains to removable stents. More particularly it relates to a delivery system and method for the placement of removable stents into urethra obstructed by enlarged prostates to alleviate restrictions in the prostatic urethra.

2. Description of Related Art

For many men, as they age, the prostate is enlarged restricting the prostatic urethra resulting in difficulties in emptying the bladder. Even slight constipation can aggravate the situation making it difficult for a person to begin urinating or emptying the bladder. A number of removable stents to relieve this condition are known.

McIntyre et al., U.S. Pat. No. 5,833,707 issued Nov. 10, 1998 discloses a removable stent for temporarily implanting an intraluminal stent in a body lumen and its subsequent removal, which operates in a manner similar to your device. McIntyre et al has an extending rolled coil design, which coils the stent into a sheath at the end of the removable catheter.

An et al., U.S. Pat. No. 6,241,757 issued Jun. 5, 2001 discloses an hour glass shaped stent with flared ends to prevent the stent from moving. Its mesh construction is used in many applications, rather than the solid stent you have in mind.

Heath, U.S. Pat. No. 7,101,392 issued Sep. 5, 2006 discloses another tubular stent made of a metal filament material with an outer member having an exposed outer surface and a core within the extended member formed of a different metal. This stent is balloon expanded and is made of a radio-opaque material to check its placement with x-rays, etc.

Reever, U.S. Pat. No. 6,790,223 issued Sep. 14, 2004 discloses a delivery system for placement of an expanding coil stent. This coil stent is dipped in liquid silicone to coat the coil segment with a webbing to prevent tissue ingrowths. A number of different types of coatings may be employed in this regard. Reever also mentions that a number of stents are positioned using an endoscope.

Robertson, U.S. Pat. No. 6,949,125 issued Sep. 27, 2005 discloses a Urethral Stent, which extends into the bladder.

Lennox et al., U.S. Pat. No. 6,494,879 issued Dec. 17, 2002 discloses a urethral prosthesis with first and second tubular elements: the first extending into the bladder into the bladder and the second holding open the urethra for relief of urinary retention.

Gellman, U.S. Pat. No. 7,527,651 issued May 5, 2009 discloses another two part stent located on either side of the external sphincter to inhibit migration while not interfering with the normal functioning of the sphincter.

The invention described below provides a practical non-intrusive stent for removing the obstruction of the flow of urine caused by enlarged prostate blockage.

### SUMMARY OF THE INVENTION

The present invention comprises a delivery system for placing a urethra stent within the urethra of a patient at the point of obstruction by an enlarged prostate. It comprises a stent and flexible stent inserter. The stent inserter has a shaft of a length to be inserted within the urethra and extend to the bladder. It has first and second ends and defines a liquid passageway there between for transporting liquids with an inlet and outlet. It also has a combination air/liquid passageway there between for transporting air/liquids with an inlet and outlet.

The first end of the stent inserter has an expandable securing segment defining an interior liquid reservoir. The liquid reservoir is in communication with the outlet of the liquid passageway to expand/contract the securing segment in response to pressure changes in the liquid passageway. The securing segment is structured to expand and hold a stent placed on the securing segment for positioning when expanded, and to contract and release the stent in place within a segment of the urethra when contracted.

A ballooning positioning tip is located proximate the securing segment and has an interior air/liquid reservoir in communication with the outlet of the combination air/liquid passageway. The positioning tip is structured to inflate/deflate with either air to form a contrast media balloon for ultrasound location and positioning of the securing segment with stent, or liquid. When inflated with liquid, the tip expands and unblocks obstructed portions of the urethra for placement of the stent.

The second end of the stent inserter contains inlets of the liquid passageway and inlets of the air/liquid passageway.

An hourglass shaped stent with an interior channel is structured to fit over the positioning tip and secured onto the securing segment. The hour glass shaped insures that the stent will remain in place in the urethra until removed. When secured, the stent is then placed within an obstructed segment of the urethra to allow urine to pass through the interior channel, when the stent inserter is removed. This stent interior channel may have rippled or roughened frictional surfaces to create better contact grip with the securing segment of the stent inserter to aid in its removal when the temporary blockage has subsided. For stents implanted longer, they may be made of an antibiotic impregnated silicone or other material resistant to bacterial adhesion. This prolongs the life of the stent by reducing tissue overgrowth.

A liquid filled inflation/deflation pump, such as a retractable syringe or squeezable bulb with an internal liquid reservoir with an opening is operably associated and is in communication with the inlet of the liquid passageway. For example, the squeeze bulb consists of a balloon made from resilient rigid material. When it is squeezed, the fluid inside is forced out. Releasing the bulb sucks fluid back in. A valve may be associated with the opening to adjust the flow of fluid, if needed.

The squeezable bulb, when compressed, forces liquid from the internal liquid reservoir through the liquid passageway to inflate the expandable securing segment to hold thereon a stent during positioning and insertion. The retractable squeezable bulb is then released to retract and deflate the securing segment to release the stent within the urethra to allow urine to pass through its interior channel.

A valve controlled air/liquid filled inflation/deflation pump, such as a retractable syringe or squeezable bulb is operably associated and in communication with the inlet of the air/liquid passageway. It has an internal reservoir initially filled with liquids and is capable of being interchanged with an internal air reservoir to selectively fill the air/liquid passageway with air. When squeezed, the air/liquid filled retractable squeezable bulb selectively forces either air from an air source to inflate the ballooning positioning tip with air in one mode or with liquids in another mode to unblock obstructed portions of the urethra for placement of a stent. After inflation, the air or liquids are withdrawn from the positioning tip, allowing removal of the stent inserter.

For example, a liquid filled squeezable air/liquid squeeze bulb with an internal liquid reservoir is in communication with the internal air/liquid passageway of the flexible inserter. A valve is included, which can selectively allow ambient air to be drawn into the internal liquid reservoir. In a sense, the air/liquid squeeze bulb is an example of a simple pump to force either air or liquids into the reservoir for pumping of gases or fluids.

The stent inserter is used by squeezing the air/liquid squeeze bulb to force first liquid from the liquid reservoir through the air/liquid passageway to inflate the ballooning positioning tip structured to force open obstructed segments of the urethra for placement of the stent. To locate where the stent is placed within the urethra near the bladder, the ballooning positioning tip is then filled with air to provide contrasting media for ultrasound location. If the stent is not at the desired location, the ballooning positioning tip is deflated and the process is repeated until the stent is located in a desired area. The ballooning positioning tip is then contracted for placement of the stent and removal of the stent inserter.

Selectively activated valves are associated with ambient air or liquid reservoirs to inflate the ballooning tip with air or liquids. Preferred valves are luer lock valves, septum vales, or push button valves, which allow either air or liquids to be selectively forced through the air/liquid passageway.

Although other contrasting media gases could be employed in the reservoirs and passageways for location of the stent, air is safe to handle and readily abundant. Similarly, other inflation liquids could be used, but water is safe to handle and readily abundant. Also, other types of air/liquid reservoirs could be used, such as plunger syringes associated with Luer lock valves.

The stent inserter system and stent thus provides a simple to use device, which allows placement without the need for a fluoroscope. In addition, as urologists usually have ultrasound equipment in their offices, and are familiar with Luer lock valves, the device avoids the need for resort to another facility to perform the stent insertion procedure. The stent inserter system and stent provides a viable, practical non-intrusive solution performed in a doctor's office, which is as simple as inserting or removing a catheter.

### DESCRIPTION OF THE DRAWINGS

FIG. 1 is a cross-sectional side view of one embodiment of the stent inserter of the invention.

FIG. 2 is a side cross-sectional view of a stent positioned with the stent inserter of FIG. 1.

FIG. 3 is a cross-sectional view of another stent positioned with the stent inserter of FIG. 1; and

FIG. 4 is an expanded cross-sectional view of the positioning tip of the stent inserter of FIG. 1.

### DESCRIPTION OF THE ILLUSTRATED EMBODIMENTS

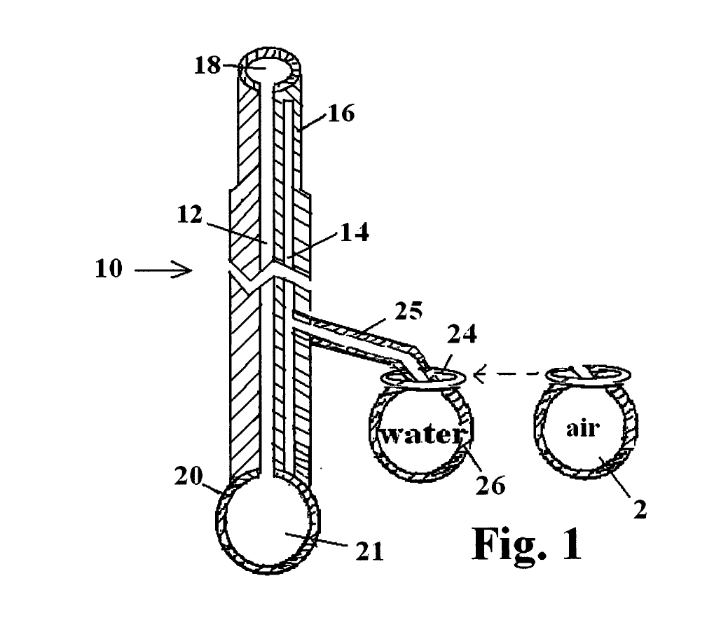

FIG. 1 is a cross-sectional side view of one embodiment of the stent inserter 10 of the invention used to position stents within obstructed segments of the urethra. The stent inserter 10 has an overall length of 409 mm. It is made of a flexible rubber, plastic or metal material with an interior liquid passageway 12 and an interior combination air/liquid passageway 14 running its length. At the end of the stent inserter 10 is an expandable stent securing end 16 operably associated with the liquid passageway 12 to expand in response to increasing hydraulic pressure therein. Next to the sent securing end 16 is a ballooning positioning tip 18 operably associated with the air/liquid passageway 14 to expand in response to increasing hydraulic or air pressure therein.

Attached to the other end of the stent inserter 10 is a squeezable bulb 20 with an interior liquid reservoir 21 in communication with the liquid passageway 12. The liquid passageway 12 is approximately 25 mm in diameter at the end connected to the squeezable bulb 20, and narrows to 4.0 mm at its upper end within the stent securing end 16. The squeezable bulb 20 is filled with water. When the bulb 20 is squeezed it creates hydraulic pressure that travels through the liquid passageway 12 increasing in pressure at the end of the narrowing liquid passageway 12 to expand the stent securing end 16 of the stent inserter 10.

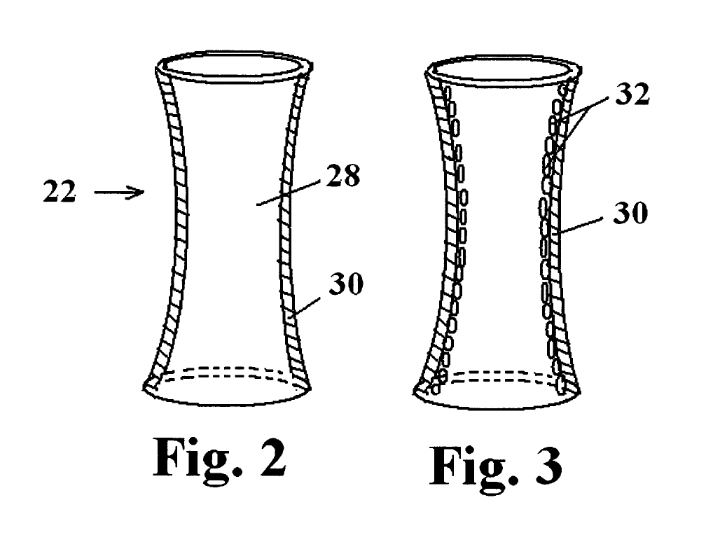

A stent 22 shown in FIGS. 2 and 3 has an hour glass shape with an interior channel 28 defined by interior walls 30. It is structured to be removably positioned on the expandable stent securing end 16 of the stent inserter 10. Pressure expansion of the securing end 20 thus holds the stent 22 firmly onto the stent securing end 16 while it is being situated in the correct position.

A Luer lock valve 24 is associated with a squeezable air or liquid bulb 27 in communication with the air/liquid passageway 14 and is initially filled with water. When it is squeezed it forces either air liquid through the combination air/liquid passageway 14 to inflate the balloon positioning tip 18. When it contracts, liquid is drawn back into its internal reservoir 27.

A second squeezable air bulb 27 is then interchanged with the liquid filled bulb 27 in communication with the air/liquid passageway 25 and 14 to inject air to inflate the positioning tip 18. The Luer Lock valve 24 mechanism manufactured by many manufacturers facilitates this interchange. Although their appearance may vary, the principle of operation is the same. The technician using same can either inject air or water. This can be accomplished by squeezing the internal reservoir 27 for water to inflate the positioning tip 18. By replacing the internal liquid filled reservoir 27 with a second air filled reservoir 27, thereby draining the system of water, the positioning tip 28 is then inflated with air. A syringe type adaptor using the valve 24 may also be used for this purpose to inflate the positioning tip 18. Inflation of the positioning tip is critical.

The inflated positioning tip 18 assists in securing the stent 22 in the right position proximate the bladder neck. The air filled positioning tip 18 creates a different media thickness that is easily detected by ultrasound equipment. This allows a doctor to place the stent 22 precisely without the use of a fluoroscope. As every urologist has access to ultrasound in his office, if the position of the stent 22 is questionable, it may be located with ultrasound and repositioned, if necessary. Water can be replaced with air entering the air/liquid passageway 14 via the Luer lock valve 24 to inflate the positioning tip 18 with air, which will show up much easier on the ultrasound than water. This gives more accurate confirmation of the positioning tip 18 and stent 22 location. The doctor therefore does not have to resort to another facility to perform the procedure using fluoroscopes.

FIG. 2 is a side cross-sectional view of a stent 22 positioned with the stent inserter 10 of FIG. 1. The stent 22, as pictured, is in its relaxed hour glass shape such as when it is left in the prostatic urethra. It has an interior channel 28 through which urine may pass. The hourglass shape keeps the stent 22 securely in position until removal. The narrowest opening of the interior channel 28 at its center is 4.0 mm. The walls 30 of the stent 22 are approximately 0.8 mm. The widest outside diameter at both ends of the stent 22 is 7.2 mm forming a hyperbolic interior shape to create the hourglass effect. The length of the stent 22 may vary depending upon the length of the obstruction within the prostate. The shortest length to solve the immediate problem is best. If the obstruction is fairly general throughout the prostate the stent 22 should be positioned 5.0 mm above the sphincter valve and protrude 4.0 mm above the top of the prostate. If the growth of the prostate or chronic inflammation occludes the stent 22, the stent 22 can be removed either with the stent inserter 10, or endoscopically.

In cases where the urethra is severely obstructed, it may require a stent 22, which is better held by the securing end 16 during positioning. FIG. 3 is a cross-sectional view of another stent with roughened or rippled surface walls 30 for better grip by the stent securing end 16.

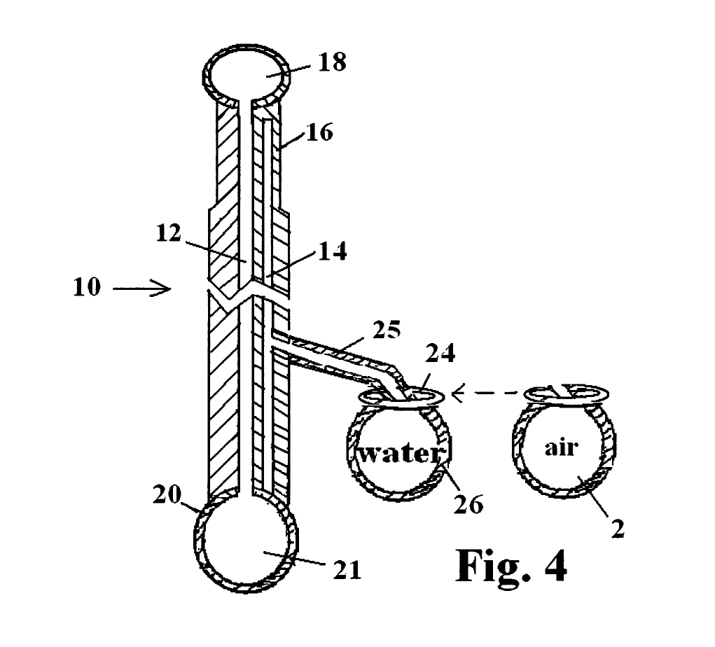

FIG. 4 is an expanded cross-sectional view of the positioning tip of the stent inserter of FIG. 1 inflated with air and the securing end 16 expanded to hold the stent 22 during positioning. The resultant water pressure from squeezable bulb 20 expands the securing end 16 and the hourglass shaped stent 22 placed thereon to the same outside diameter as the stent inserter 10, which is 6.4 mm for ease of insertion.

When stent 22 is in the desired location Luer lock valve 24, is then set for air injection and is squeezed. Squeezing the Luer lock valve 24 forces air through the air/liquid passageway 16 inflating the positioning tip 18 for ultrasound positioning. Pressing Luer lock valve 24 again releases the air pressure and deflates the positioning tip 18 for easy removal of the stent inserter 10.

It is estimated that this device will provide relief for over 65% of the patients with urination difficulties, which require frequent trips to the bathroom to empty the bladder. The fact that this can be accomplished in a urologist's office without having to go to an emergency room out patient clinic saves expense, and better serves patient's needs.

Although this specification has referred to the specific embodiments, it is not intended to restrict the scope of the claims. The scope of the invention is, therefore, indicated by the appended claims, rather than by the foregoing description. All changes that come within the meaning and range of equivalency of the claims are to be embraced within their scope.

1. A device for placing, positioning and/or removing a stent within the urethra of a patient obstructed by an enlarged prostate positioned by use of ultrasound comprising:
a. a flexible stent inserter having
i. a shaft of a length and size to be inserted within the urethra and extend to the bladder, with first and second ends defining a liquid passageway for transporting liquids with an inlet and outlet and a combination air/liquid passageway for transporting air/liquids with an inlet and outlet, and
ii. a first end with
I. an expandable securing segment with an interior liquid reservoir in communication with the outlet of the liquid passageway to expand/contract in response to pressure changes in the liquid passageway; the securing segment structured to expand and hold a stent placed on the securing segment for positioning when expanded, and to contract and release the stent in place within a segment of the urethra when contracted, and
II. a ballooning positioning tip distally mounted proximate the securing segment with an air/liquid reservoir in communication with the outlet of the combination air/liquid passageway to either inflate/deflate with either air to form a contrast media for ultrasound location and positioning of the proximate securing segment with stent, or liquid to expand and unblock obstructed portions of the urethra before placement of the stent within the unblocked portion; and
iii. a second end with inlets of the liquid passageway and inlets of the air/liquid passageway;
b. an hourglass shaped stent with interior walls defining an interior channel structured to fit over the positioning tip and onto the securing segment, which when positioned within an obstructed segment of the urethra allows urine to pass through the interior channel.
c. an inflation/deflation pump with an internal liquid reservoir in communication with the inlet of the liquid passageway, which, when activated in a first mode, forces liquid from the liquid reservoir to inflate the expandable securing segment to hold the stent during positioning and insertion, and in a second mode deflates the securing segment to release the stent within the urethra, and
d. a valve controlled inflation/deflation pump in communication with the inlet of the air/liquid passageway having an internal reservoir selectively fillable with either liquids or air, which when activated in a first mode forces either air from an air source to inflate the ballooning positioning tip, or liquids from a liquid source to unblock obstructed portions of the urethra for placement of a stent, and in a second mode deflates the ballooning positioning tip to allow the flexible stent inserter to be withdrawn and allow urine to pass through the stent.

2. A device for placing, positioning, and/or removing a stent according to claim 1, wherein the stent interior channel has walls with a frictional surface to create better contact with the securing segment of the inserter to aid in removal.

3. A device for placing, positioning, and/or removing a stent according to claim 1, wherein the stent is constructed of a material resistant to bacterial adhesion.

4. A device for placing, positioning, and/or removing a stent according to claim 1, wherein the inflation/deflation pump is a liquid filled squeezable bulb with an internal liquid reservoir in communication with the inlet of the liquid passageway, which, when squeezed, forces liquid from the liquid reservoir to inflate the expandable securing segment to hold the stent during positioning and insertion, and to deflate the securing segment to release the stent within the urethra when unsqueezed, and the valve controlled inflation/deflation pump is a valve controlled air/liquid filled squeezable bulb in communication with the inlet of the air/liquid passageway having an internal reservoir initially filled with liquids and selectively fillable with air, which when squeezed forces either air from an air source to inflate the ballooning positioning tip, or liquids to unblock obstructed portions of the urethra for placement of a stent.

5. A device for placing, positioning, and/or removing a stent according to claim 4, wherein the air/liquid filled squeezable bulb is initially filled with liquid, and includes a valve associated with ambient air to allow a second air filled reservoir to be selectively connected so that air is pumped into the air/liquid passageway to inflate the balloon positioning tip creating contrasting media for ultrasound location to position the stent in one mode, and to release air to deflate the balloon positioning tip to remove the inserter in another mode.

6. A device for placing, positioning, and/or removing a stent according to claim 5, wherein the valve is selected from the group of a luer lock valve, a septum valve, and a push button valve.

7. A device for placing, positioning, and/or removing a stent according to claim 1, wherein the liquid passageway has a longitudinal decreasing cross sectional area with its widest cross section area proximate the squeezable bulb and its narrowest cross-sectional area proximate the securing segment to increase hydraulic pressure at the securing segment, when the inflation deflation pump is activated.

8. A method for placing, positioning, and/or removing a stent within a prostate obstructed segment of a urethra positioned by use of ultrasound comprising:
a. employing a delivery system for placing a stent within the urethra of a patient obstructed by an enlarged prostates having:
a(1). a flexible stent inserter with
a(1)(i). a shaft of a length and size to be inserted within the urethra and extend to the bladder, with first and second ends defining a liquid passageway for transporting liquids with an inlet and outlet and a combination air/liquid passageway for transporting air/liquids with an inlet and outlet, and
a(1)(ii). a first end with
a(1)(ii)(I). an expandable securing segment with an interior liquid reservoir in communication with the outlet of the liquid passageway to expand/contract in response to pressure changes in the liquid passageway; the securing segment structured to expand and hold a stent placed on the securing segment for positioning when expanded, and to contract and release the stent in place within a segment of the urethra when contracted, and
a(1)(ii)(II). a ballooning positioning tip distally mounted proximate the securing segment with an air/liquid reservoir in communication with the outlet of the combination air/liquid passageway to either inflate/deflate with either air to form a contrast media for ultrasound location and positioning of the proximate securing segment with stent, or liquid to expand and unblock obstructed portions of the urethra before placement of the stent within the unblocked portion; and
a(1)(iii). a second end with inlets of the liquid passageway and inlets of the air/liquid passageway;
a(2). an hourglass shaped stent with an interior channel structured to fit over the positioning tip and onto the securing segment, which when positioned within an obstructed segment of the urethra allows urine to pass through the interior channel.
a(3). an inflation/deflation pump with an internal liquid reservoir in communication with the inlet of the liquid passageway, which, when activated in a first mode, forces liquid from the liquid reservoir to inflate the expandable securing segment to hold the stent during positioning and insertion, and in a second mode deflates the securing segment to release the stent within the urethra when unsqueezed, and
a(4). a valve controlled inflation/deflation pump in communication with the inlet of the air/liquid passageway having an internal reservoir selectively fillable with either liquids or air, which when activated in a first mode forces either air from an air source to inflate the ballooning positioning tip, or liquids from a liquid source to unblock obstructed portions of the urethra for placement of a stent, and in a second mode deflates the ballooning positioning tip to allow the flexible stent inserter to be withdrawn and allow urine to pass through the stent.
b. placing a stent on the positioning segment of the securing segment and securing it thereon by activating the inflation/deflation pump;
c. positioning the positioning tip proximate the bladder after inflation with air from activating the valve controlled inflation/deflation pump and using ultrasound to verify proper positioning, and
d. releasing the stent within the urethra by deflating the positioning tip and securing segment, and withdrawing the flexible stent inserter.

9. A method for placing, positioning, and/or removing a stent within a prostate obstructed segment of a urethra according to claim 8, wherein the inflation/deflation pump is a liquid filled squeezable bulb with an internal liquid reservoir in communication with the inlet of the liquid passageway, which, when squeezed, forces liquid from the liquid reservoir to inflate the expandable securing segment to hold the stent during positioning and insertion, and to deflate the securing segment to release the stent within the urethra when unsqueezed, and the valve controlled inflation/deflation pump is a valve controlled air/liquid filled squeezable bulb in communication with the inlet of the air/liquid passageway having an internal reservoir initially filled with liquids and selectively fillable with air, which when squeezed forces either air from an air source to inflate the ballooning positioning tip, or liquids to unblock obstructed portions of the urethra for placement of a stent.
